(19) United States
(12) Patent Application Publication
(10) Pub. No.: US 2009/0326619 A1
(43) Pub. Date: Dec. 31, 2009

(54) HOT/COLD REUSABLE THERAPUTIC PACK

(76) Inventor: Stanislav Kagan, Granada Hills, CA (US)

Correspondence Address: VLADIMIR KHITERER;KHITERER LAW OFFICE, 2901 W. COAST HWY., SUITE 200, NEWPORT BEACH, CA, 92663, US

(21) Appl. No.: 12/145,878

(22) Filed: Jun. 25, 2008

(51) Int. Cl. A61F 7/00; A61H 1/00

(52) U.S. Cl. 607/96; 601/15

(57) ABSTRACT

A hot/cold reusable therapeutic pack is disclosed. The pack comprises a first pouch with salt and a second pouch with sand disposed on top of the first pouch. The pack provides good heat/cold retention and ability to evacuate fluids from the affected area.

### BACKGROUND OF THE INVENTION

This invention relates to a hot/cold reusable therapeutic pack and specifically the pack that utilizes salt and sand, or mixture thereof, as heat storage and moisture absorbing substances.

Application of hot and/or cold to a patient by way of compresses or packs has been used for therapeutic purposes for centuries. Such remedies remain a standard medical practice for reducing inflammation and pain. In essence, application of heat and cold causes increasing blood flow to various parts of the anatomy. Application of cold is generally recommended shortly after an injury, such as a sprain, to reduce swelling and retard inflammation. Application of heat is recommended thereafter due to its desirable therapeutic effects, such as increasing the extensibility of the collagen tissue; decreasing joint stiffness; pain relief; relieving muscle spasms; reducing inflammation and edema; increasing blood flow.

Conventional hot/cold therapy packs use jelly-like moist material, or other material capable of good heat retention, that can be heated or cooled and applied to the affected area. However, it may be desirable to evacuate moisture and fluids from a swollen area, which cannot be accomplished by jelly-based and the like hot/cold therapy packs.

### SUMMARY OF THE INVENTION

This invention provides an improved hot/cold reusable therapeutic pack that has both good heat/cold retention and ability to evacuate fluids from the affected area. In one embodiment, the hot/cold reusable therapeutic pack comprises a first pouch applied to the affected area filled with salt and a second pouch on top of the first pouch filled with sand. The grains of sand are larger than the grains of salt. Moisture from the affected area is absorbed by salt and then absorbed by sand from salt, and then evacuated from sand to the air by way of evaporation.

Further, the specific heat capacity of salt is slightly different from that of sand, resulting in slightly different cooling rates between sand and salt. This causes a tempering effect on the hot/cold pack.

In another embodiment, the hot/cold reusable therapeutic pack comprises a pouch applied to the affected area filled with a mixture of substantially 50% salt and 50% sand. Similarly, the grains of sand are larger than the grains of salt. Moisture from the affected area is absorbed by salt/sand mixture and then evacuated from the salt/sand mixture to the air by way of evaporation. The salt/sand mixture also provides a tempering effect on the hot/cold pack.

### BRIEF DESCRIPTION OF THE DRAWINGS

FIG. 1 is a cross-sectional view of the hot/cold reusable therapeutic pack according to the first embodiment of this invention.

FIG. 2 is a cross-sectional view of the hot/cold reusable therapeutic pack according to the second embodiment of this invention.

FIG. 3 is a perspective view of the hot/cold reusable therapeutic pack according to the second embodiment of this invention.

### DETAILED DESCRIPTION

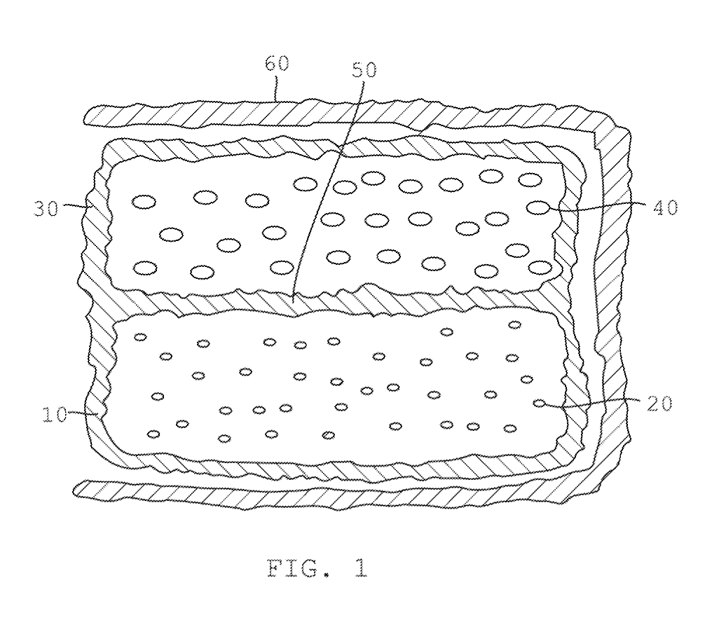

This invention will be better understood with the reference to the drawing figures FIG. 1 through FIG. 3. The same numerals refer to the same elements in all drawing figures.

Viewing now, FIG. 1, numeral 10 indicates a first sealed pouch. First sealed pouch 10 is applied to the affected area of a patient, for example, swelling caused by a sprain. First sealed pouch 10 comprises a first granular material indicated by numeral 20.

Numeral 30 indicates a second sealed pouch. Second sealed pouch 30 is disposed on top of first sealed pouch 10. Second sealed pouch 30 is shaped and sized substantially identically to first sealed pouch 10. Second sealed pouch 30 comprises a second granular material indicated by numeral 40.

Numeral 50 indicates a membrane. Membrane 50 separates first sealed pouch 10 and second sealed pouch 30. Membrane 50, first sealed pouch 10 and second sealed pouch 30 are made from a liquid permeable fabric, which is in this embodiment cotton but may be any other liquid permeable fabric known to the persons most knowledgeable in the pertinent arts. Cotton has excellent moisture absorbing properties and prevents dust from first granular material 20 and second granular material 40 from coming out.

Second granular material 40 comprises granules that are larger than granules comprising first granular material 20. In this embodiment, first granular material 20 is salt and second granular material 40 is sand. Only salt that can be heated by microwave and basic oven and does not harden or melt is appropriate for this embodiment. Salt selected from the group comprising sodium chloride salt and sodium silicoaluminate salt is perfect for this embodiment and other similar salts may be used.

For heat application, the hot/cold reusable therapeutic pack is heated. A microwave can be used for heating. For cold application, the hot/cold reusable therapeutic pack is cooled. A freezer can be used for cooling. In either case, the hot/cold reusable therapeutic pack is applied to the affected area with first sealed pouch 10.

In this embodiment, the grains of sand in second sealed pouch 30 are larger than the grains of salt in first sealed pouch 10. Moisture from the affected area is absorbed by salt and then absorbed by sand from salt, and then evacuated from sand to the air by way of evaporation. Further, the specific heat capacity of salt is slightly different from that of sand, resulting in slightly different cooling rates between sand and salt. This causes a tempering effect on the hot/cold pack.

Numeral 60 indicates a removable outer case. Removable outer case 60 envelops first sealed pouch 10 and second sealed pouch 30. Removable outer case 60 is made from cotton and is removed and washed after each use.

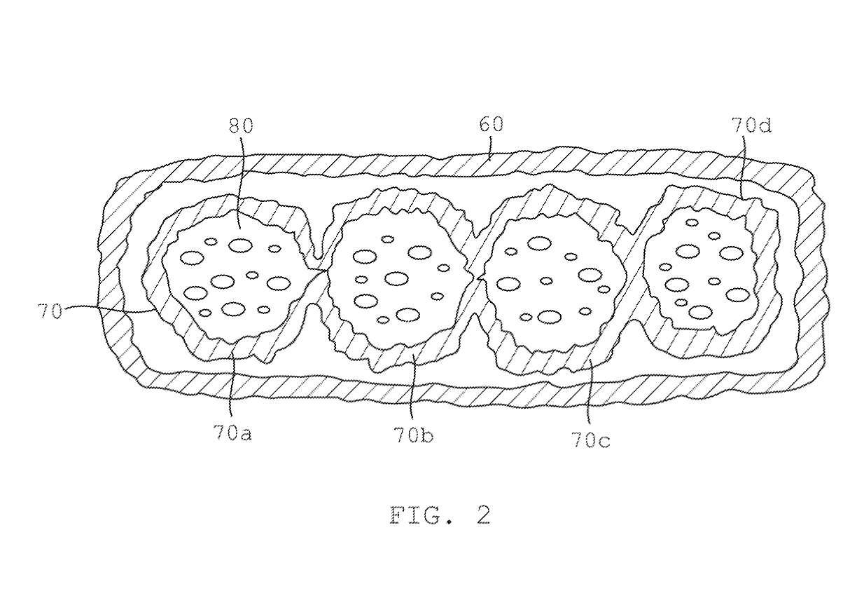

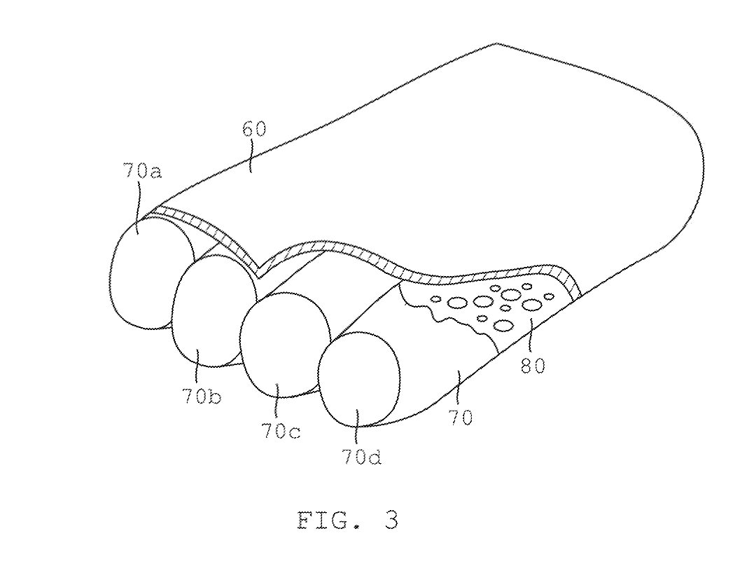

Viewing now, simultaneously, FIG. 2 and FIG. 3, numeral 70 indicates a sealed pouch. Sealed pouch 70 is applied to the affected area of a patient, for example, swelling caused by a sprain. Sealed pouch 70 comprises a mixture of substantially 50% of a first granular material and substantially 50% of a second granular material indicated by numeral 80. Granules comprising the second granular material are larger than granules comprising the first granular material. In this embodiment, the first granular material comprising mixture 80 is salt and second granular material comprising mixture 80 is sand. This mixture of salt and sand in one pouch works similarly to separate pouches of salt and sand described with respect to the first embodiment. In this embodiment, sealed pouch 70 is made from a liquid permeable fabric, which is in this embodiment cotton but may be any other liquid permeable fabric known to the persons most knowledgeable in the pertinent arts.

In this embodiment, sealed pouch 70 is partitioned into a plurality of cavities. In FIGS. 3 and 4 there are four cavities indicated by numerals 70a, 70b, 70c and 70d. Such partitioning of sealed pouch 70 into cavities 70a, 70b, 70c and 70d allows the hot/cold reusable therapeutic pack to better maintain its shape.

While the present invention has been described and defined by reference to the preferred embodiment of the invention, such reference does not imply a limitation on the invention, and no such limitation is to be inferred. The invention is capable of considerable modification, alteration, and equivalents in form and function, as will occur to those ordinarily skilled and knowledgeable in the pertinent arts. The depicted and described preferred embodiments of the invention are exemplary only, and are not exhaustive of the scope of the invention. Consequently, the invention is intended to be limited only by the spirit and scope of the appended claims, giving full cognizance to equivalents in all respects.

What is claimed is:

1. A hot/cold reusable therapeutic pack for delivering heat or cold to a selected body part of a human or animal comprising:
(a) a first sealed pouch for applying to the body part, the first pouch comprising a first granular material, the first granular material comprising granules of a first size;
(b) a second sealed pouch disposed on top of the first sealed pouch, the second sealed pouch shaped and sized substantially identically to the first sealed pouch, comprising a second granular material, the second granular material comprising granules of a second size;
(c) a membrane separating the first sealed pouch and the second sealed pouch;
wherein the first sealed pouch, the second sealed pouch and the membrane are made from a liquid permeable fabric;
wherein the second size is larger than the first size.

2. A hot/cold reusable therapeutic pack as in claim 1, wherein liquid permeable fabric is cotton.

3. A hot/cold reusable therapeutic pack as in claim 2, wherein the first granular material is salt and the second granular material is sand.

4. A hot/cold reusable therapeutic pack as in claim 3, wherein the salt is selected from the group comprising sodium chloride salt and sodium silicoaluminate salt.

5. A hot/cold reusable therapeutic pack as in claim 4, further comprising a removable outer case enveloping the first sealed pouch and the second sealed pouch made from cotton.

6. A hot/cold reusable therapeutic pack for delivering heat or cold to a selected body part of a human or animal comprising:
(a) a sealed pouch for applying to the body part comprising a mixture of substantially fifty percent of a first granular material, the first granular material comprising granules of a first size, and substantially fifty percent of a second granular material, the second granular material comprising granules of a second size;
wherein the sealed pouch is made from a liquid permeable fabric;
wherein the second size is larger than the first size.

7. A hot/cold reusable therapeutic pack as in claim 6, wherein liquid permeable fabric is cotton.

8. A hot/cold reusable therapeutic pack as in claim 7, wherein the first granular material is salt and the second granular material is sand.

9. A hot/cold reusable therapeutic pack as in claim 8, wherein the salt is selected from the group comprising sodium chloride salt and sodium silicoaluminate salt.

10. A hot/cold reusable therapeutic pack as in claim 9, wherein the sealed pouch is partitioned into a plurality of cavities.

11. A hot/cold reusable therapeutic pack as in claim 10, further comprising a removable outer case made from cotton enveloping the sealed pouch.
